(19) United States
(12) Patent Application Publication
(10) Pub. No.: US 2023/0022665 A1
(43) Pub. Date: Jan. 26, 2023

(54) PRESHAPED BIOLOGIC SCAFFOLDS

(71) Applicant: Stephane Gobron, San Marino, CA (US)

(72) Inventors: Robert Greer, Cooper City, FL (US); Ty Erickson, Henderson, NV (US); Stephane Gobron, San Gabriel, CA (US)

(73) Assignee: ARMS Inc., Sunrise, CA (US)

(21) Appl. No.: 17/689,661

(22) Filed: Mar. 8, 2022

Related U.S. Application Data
(63) Continuation of application No. 63/225,522, filed on Jul. 25, 2021.

(51) Int. Cl. A61F 2/00; A61B 17/42

(52) U.S. Cl. CPC A61F 2/0004; A61F 2/0063; A61B 17/42; A61B 2017/00805

(57) ABSTRACT

The present invention relates to biologic tissues, which are used to create cellular matrix scaffolds to help repair the body. The invention discloses two specific implant shapes that are pre-cut to address two separate pelvic floor prolapses (anterior and/or posterior). Additionally, the method of providing surgeries-specific pre-shaped biologic scaffolds is also claimed.

### BACKGROUND

### Field of the Invention

This invention relates to compositions and methods for generating a biological scaffold that can be used, for example, in tissue engineering and in particular to systems, devices, and methods for designing and producing pre-shaped biologic scaffolds to repair various body areas.

### Description of the Related Art

Biologic tissues have been used to create cellular matrix scaffolds to help the body repair a generally weak area. Tissue sources from which biologic scaffolds are chosen include both allografts, such as human-donated tissue, and xenograft, such as porcine (small intestine, dermis), bovine (pericardium, fetal, dermis), or equine tissues.

Naturally occurring extracellular matrices (ECMs) are used for tissue repair and regeneration. One such extracellular matrix is the small intestine submucosa (SIS). SIS has been used to repair, support, and stabilize various anatomical defects and traumatic injuries. Commercially-available SIS material is derived from porcine small intestinal submucosa that remodels to the qualities of its host when implanted in human soft tissues. Further, it is thought that the SIS material provides a natural matrix with a three-dimensional microstructure and biochemical composition that facilitates host cell proliferation and supports tissue remodeling. Indeed, SIS has been shown to contain biological molecules, such as growth factors and glycosaminoglycans, that aid in repairing the soft tissue of the human body. The SIS material currently being used in the orthopedic field is provided in a dried and layered configuration in a patch to repair or regenerate soft tissue such as tendons, ligaments, and rotator cuffs.

While small intestine submucosa is readily available, other sources of ECM are known to be effective for tissue remodeling. These sources include but are not limited to stomach, bladder, alimentary, respiratory, genital submucosa, or liver basement membrane. See, e.g., U.S. Pat. Nos. 6,379,710, 6,171,344; 6,099,567; and 5,554,389, each of which is hereby incorporated by reference.

Typically, the tissues are decellularized to remove DNA and other genetic materials and are provided in a generally rectangular shape. After the surgeon evaluates the specific requirements during surgery, the surgeon must cut the tissue into the desired shape. The manipulation of scaffold pore size, porosity, and interconnectivity is a vital science contributing to the field of tissue engineering (Ma and Zhang, 2001, J Biomed Mater Res, 56(4):469-477; Ma and Choi, 2001 Tissue Eng, 7(1):23-33) because it is believed that the consideration of scaffold pore size and density/porosity influences the behavior of cells and the quality of tissue regenerated. Several researchers have shown that different pore sizes influence the behavior of cells in porous three-dimensional matrices. For example, it has been demonstrated in the art that for adequate bone regeneration to occur, scaffold pore size needs to be at least 100 microns (Klawitter et al., 1976, J Biomed Mater Res, 10(2):311-323). Poor quality bone is regenerated for pore sizes and interconnectivity less than that. If the pore size is between 10-40 microns, bone cells can form only soft fibro-vascular tissue (White and Shors, 1991 Dent Clin North Am, 30:49-67). The consensus of research for bone regeneration indicates that the requisite pore size for bone regeneration is 100-600 microns (Shors, 1999, Orthop Clin North Am, 30(4):599-613; Wang, 1990, Nippon Seikeigeka Gakki Zasshi, 64(9):847-859). It is generally known in the art that optimal bone regeneration occurs for pore sizes between 300-600 microns.

Considerable advancements can be seen in the prior art in this regard. For instance, U.S. Pat. No. 7,319,035B2 provides methods of generating a natural, living biological matrix that can serve as, or form a part of, a natural biological scaffold. The matrix is generated by incubating together biological cells and cellular debris. Any naturally occurring cell can be a biological cell included in the matrix. The invention also provides methods of treating a patient by implanting the matrix or scaffolding into the patient's tissue, thereby augmenting the existing tissue.

U.S. Pat. No. 9,114,196B2 provides a method for producing a bioscaffold from natural tissues by oxidizing a decellularized tissue to produce a bioscaffold with pores therein. The pore size and porosity are increased to accommodate intact cells better so that live cells can better infiltrate and inhabit the bioscaffold. The bioscaffold may be freeze-dried or lyophilized, sterilized, and (optionally) aseptically packaged for subsequent use. A further aspect of the present invention is a bioscaffold produced by the described processes. Methods of treatment using the bioscaffold as a graft or a biomedical implant for implantation are also provided. Also provided are methods of seeding a bioscaffold with mammalian cells, wherein the seeding is carried out either in vitro or in vivo. A bioscaffold produced as described herein is utilized for said seeding.

US patent 2,002,0119437A1 discloses a method for processing collagen-based tissues or organs to substantially decellularize said collagen-based tissues or organs before implantation into a recipient in need thereof. Preferably, the method significantly reduces or eliminates immunogenicity of the tissue or organ such that upon transplantation, the tissue or organ is not rejected by the recipient's immune system. The method includes removing the tissue from a donor, processing the tissue to remove all tissue or organ cells substantially, and processing the collagen scaffold for storage. The method further includes repopulating the collagen scaffold through seeding with stem cells for implantation into the recipient.

U.S. Pat. No. 7,108,721B2 is based on discovering new methods for obtaining a tissue graft. The technique can be carried out, for example, by implanting a biocompatible scaffold into a mammal and then removing the scaffold from the mammal. The scaffold is implanted in direct contact with (i.e., physically touching over at least a portion of its external surface) or adjacent to (i.e., physically separated from the) mature or immature target tissue, for a period that is sufficient to allow cells of the target tissue to associate with the scaffold. The invention also features grafts generated by the methods described herein and methods for using those grafts to treat patients who have a defect in a target.

U.S. Pat. No. 7,201,917B2 based on a method of making an implantable scaffold for repairing damaged or diseased tissue includes the step of suspending pieces of extracellular matrix material in a liquid. The extracellular matrix material and the liquid are formed into a mass. The liquid is subsequently driven off to form interstices in the mass. The scaffold may further comprise biological agents that promote tissue repair and healing. Porous implantable scaffolds fabricated by such a method are also disclosed.

It must be noted that the current invention proposes an advancement where it provides a new approach of a system, devices, and methods for designing and producing pre-shaped biologic scaffold to repair various areas of the body. More specifically, this invention is beneficial for surgeries, where the overall shapes can be defined pre-cut after the tissue is processed, packaged, and sold to surgeons. The surgeons bypass the implant cutting step during surgery, resulting in a shorter surgery duration. Additionally, as the shapes are defined, it offers the potential to optimize the cutting to create more implants from a single piece of tissue, increasing overall yield from a precious limited-supply raw material such as donated human tissues.

None of the previous inventions and patents, taken either singly or in combination, describes the instant invention as claimed, Hence, the inventor of the present invention proposes to resolve and surmount existent technical difficulties to eliminate the shortcomings mentioned above of prior art,

### SUMMARY

In light of the disadvantages of the prior art, the following summary is provided to facilitate an understanding of some of the innovative features unique to the present invention and is not intended to be a complete description. A full appreciation of the various aspects of the invention can be gained by taking the entire specification, claims, drawings, and abstract as a whole.

The present invention seeks to improve upon prior biologic scaffolds to repair various body areas.

Another object of the present invention is to provide two specific shapes to address pelvic organ prolapse; one is specific to the anterior prolapse and the other to the posterior prolapse.

It is a more particular object of the present invention to provide an improved system that eliminates trials and errors by giving pre-defined shapes for specific surgeries.

It is also the object of the invention to disclose two specific shapes. These designs offer the ability to adjust the length of the implant by the surgeon by having an easy one-cut across the bottom of the shape.

It is further the objective of the invention to provide an assembly that aims to reduce the time of the operation and minimize errors and wastage.

It is also the objective of the invention to provide an assembly that increases control over the surgical site and enhances both the speed and quality of the entire procedure

It is also the objective of the invention to provide a whole construction that is compatible with high-volume manufacturing methods for all components. It exposes a broader public audience to first-hand scientific advancements.

Thus, it is the objective to provide a new and improved:form of advancement in producing a pre-shaped biologic scaffold to repair various body areas. Other aspects, advantages, and novel features of the present invention will become apparent from the detailed description when considered in conjunction with the accompanying drawings.

This summary is provided merely to summarize some example embodiments to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter are described herein.

### BRIEF DESCRIPTION OF THE DRAWINGS

The accompanying figures, where like reference numerals refer to identical or functionally similar elements throughout the separate views, together with the detailed description below, are incorporated in and form part of the specification and serve to illustrate further embodiments of concepts that include the claimed invention and explain various principles and advantages of those embodiments.

FIG. 1 is a front view of anterior pelvic prolapse implant cut out showing the new design.

FIG. 2 is a front view of a posterior view of pelvic prolapse implant cut out showing the new design.

Skilled artisans will appreciate that elements in the figures are illustrated for simplicity and clarity and have not necessarily been drawn to scale. For example, the dimensions of some of the features in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. However, it is to be understood that the present invention may be embodied in various forms. Therefore, specific details disclosed herein are not to be interpreted as limiting but rather as a basis for the claims and a representative basis for teaching one skilled in the art to employ the present invention in virtually any appropriately detailed system, structure, or manner.

The terminology used herein describes particular embodiments only and is not intended to limit the invention. As used herein, the term “and” includes any combinations of one or more of the associated listed items. As used herein, the singular forms “a,” “an,” and “the” are intended to include the plural forms as well as the singular forms unless the context indicates otherwise. It will be further understood that the terms “comprises” and “comprising,” when used in this specification, specify the presence of stated features, steps, operations, elements, and components but do not preclude the presence or addition of one or more other features, steps, operations, elements, components, and/or groups thereof.

The present invention relates to pre-shaped biological materials for implant applications. The process involves manufacturing/processing the biological product to pre-cut to a shape specific to a particular type of application prior to final packaging for commercial distribution.

The present invention is based on discovering new methods for obtaining a tissue graft (which may also be referred to herein as an “implant”). The method can be carried out, for example, by implanting a biocompatible scaffold (i.e., a three-dimensional, partially porous structure that fails to cause an acute reaction when implanted into a patient) into a mammal. The scaffold is implanted in direct contact with (i.e., physically touching over at least a portion of its external surface) or adjacent to (i.e., physically separated from the) mature or immature target tissue, for a period that is sufficient to allow cells of the target tissue to associate with the scaffold. The tissue graft includes the removed scaffold and the associated cells of the target tissue.

The biological tissues from various origins (human, animal, porcine, bovine, equine, or from fish) are created to create the implant in multiple application types (hernia, or pelvic organ prolapse). These biologics are processed to remove various components to ensure safety. Currently, these biologics are sent with either “as harvested” shape for human donor tissues or in a generally rectangular shape; the surgeon then needs to cut the implant to shape it for its particular application.

After the surgeon evaluates the specific requirements during surgery, the surgeon must cut the tissue into the desired shape. The surgeons bypass the implant cutting step during surgery, resulting in a shorter surgery duration.

Additionally, as the shapes are defined, it offers the potential to optimize the cutting to create more implants from a single piece of tissue, increasing overall yield from a precious limited-supply raw material such as donated human tissues.

As per its preferred embodiments, the current invention discloses a novel method of pre-shaped biologics material specifically for the anterior or posterior pelvic organ prolapse using pre-shaped biologics for implant applications.

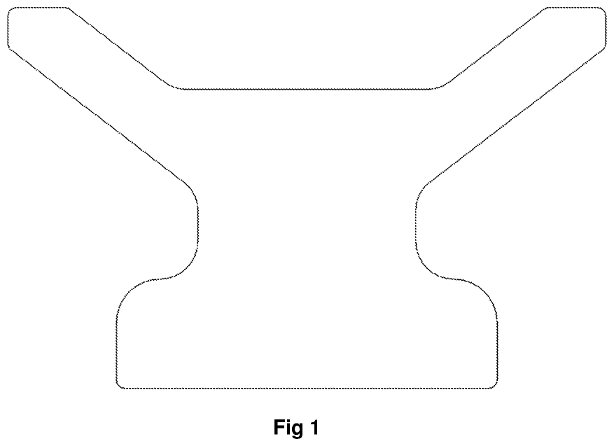

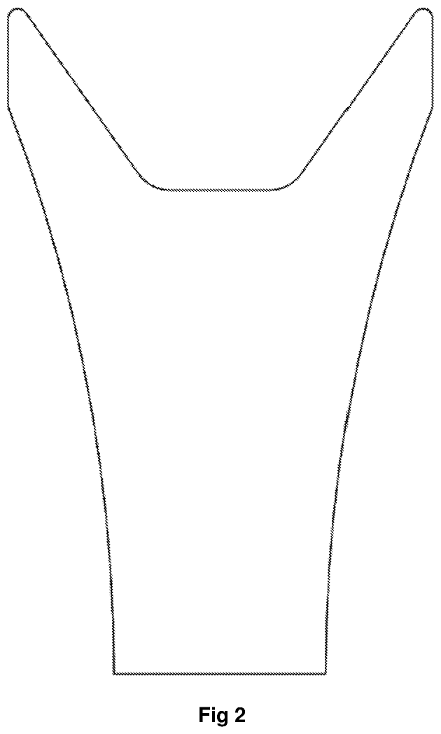

By referring to figures, FIG. 1 shows the shape of an anterior pelvic prolapse implant cut out of biologics material by the manufacturer before packaging. FIG. 2 shows the shape of a posterior pelvic prolapse implant cut out of biologics material by the manufacturer before packaging.

The assembly is developed from biological sources. Tissue sources from which biologic scaffolds are chosen include both allografts, such as human-donated tissue, and xenograft, such as porcine (small intestine, dermis), bovine (pericardium, fetal, dermis), equine, or from fish tissues. Typically, the tissues are decellularized to remove DNA and other genetic materials and are provided in a generally rectangular shape.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to it without departing from its spirit. Therefore, it is not intended that the scope of the invention be limited to the specific embodiment illustrated and described. Instead, it is intended that the appended claims and their equivalents determine the scope of this invention.

The Abstract of the Disclosure is provided to allow the reader to ascertain the technical disclosure's nature quickly. It is submitted with the understanding that it will not be used to interpret or limit the scope or meaning of the claims. In addition, in the preceding Detailed Description, it can be seen that various features are grouped in various embodiments to streamline the disclosure. This method of disclosure is not to be interpreted as reflecting an intention that the claimed embodiments require more features than are expressly recited in each claim. Instead, as the following claims reflect, the inventive subject matter lies in less than all features of a single disclosed embodiment. Thus, the following claims are hereby incorporated into the Detailed Description, with each claim standing on its own as a separately claimed subject matter.

We claim:

1. A pre-shaped biologic scaffold to repair various pelvic conditions comprising:
a surgical implant, cut out of biological material, pre-cut with a shape suitable for a specific clinical purpose prior to packaging, wherein the shape is suitable for treating vaginal prolapse.

2. A surgical implant of claim 1, wherein the biological material undergoes a series of processing steps prior to packaging.

3. A surgical implant of claim 1, wherein the biological material is of human origins, such as donated or harvested human tissues.

4. A surgical implant of claim 1, wherein the biological material is of animal origin, such as, but not limited to, porcine, bovine, equine, or from fish tissues.

5. A surgical implant of claim 1, wherein the biological material is originated or derived from human, animal, or plant tissues.

6. A surgical implant of claim 1, wherein the specific shape is intended to address anterior pelvic organ prolapse.

7. A surgical implant of claim 1, wherein the specific shape is intended to address posterior pelvic organ prolapse.

8. A systems, devices, and methods for designing and producing pre-shaped biologic scaffold to repair various areas of the body comprising:
a surgical implant, cut out of biological material, pre-cut with a shape suitable for a specific clinical purpose before final packaging.

9. A surgical implant of claim 8, wherein the biological material undergoes a series of processing steps prior to final packaging.

10. A surgical implant of claim 8, wherein the biological material is of human origin.

11. A surgical implant of claim 8, wherein the biological material is of animal origin, such as, but not limited to, porcine, bovine, equine, or:from fish origin.

12. A surgical implant of claim 8, wherein the biological material is derived from cells of biological origin such as human, animal, or plant.

13. A surgical implant of claim 8, wherein the specific shape is intended to address anterior pelvic organ prolapse.

14. A surgical implant of claim 8, wherein the specific shape is intended to address posterior pelvic organ prolapse.

15. A method of treating a patient, the method comprising:
providing a surgical implant made of tissues of biological origin, the implant having a shape particularly suited for a specific surgery right out of its packaging,
surgically implanting the implant.

16. A surgical implant of claim 15, wherein the tissues undergo a series of processing steps prior to final packaging.

17. A surgical implant of claim 15, wherein the tissues are derived from biological cells from human, animal, or plant origins.

18. The method of claim 15, wherein the implant is partially rimmed during the surgery, before, during, or after implantation.

19. The method of claim 15, wherein the method comprises treating a pelvic condition.

20. The method of claim 15, wherein the method comprises treating a vaginal prolapse condition.
